(19) United States
(12) Patent Application Publication
(10) Pub. No.: US 2026/0018256 A1
(43) Pub. Date: Jan. 15, 2026

(54) AN ODOR PREDICTION METHOD FOR AQUEOUS POLYMER COMPOSITION

(71) Applicants: DOW GLOBAL TECHNOLOGIES LLC, Midland, MI (US); ROHM AND HAAS COMPANY, Collegeville, PA (US); DOW SILICONES CORPORATION, Midland, MI (US)

(72) Inventors: Han LYU, Shanghai (CN); Yan MA, Shanghai (CN); Chenyi SHI, Shanghai (CN); Jianming XU, Shanghai (CN); Jian ZOU, Shanghai (CN); Zhouhua JI, Shanghai (CN); Jonathan DEROCHER, Coopersburg, PA (US)

(21) Appl. No.: 18/992,656

(22) PCT Filed: Aug. 2, 2022
(86) PCT No.: PCT/CN2022/109691

§ 371 Date: Jan. 9, 2025

(51) Int. Cl. G16C 20/30; G16C 20/70

(52) U.S. Cl. CPC G16C 20/30; G16C 20/70

(57) ABSTRACT

A method and a system (400) for predicting odor of an aqueous polymer composition, such as a polymerising coating, comprising: analytically characterizing the aqueous polymer composition with a detector (4013), thereby generating concentration data for volatile organic compounds in the aqueous polymer composition from the analytical characterization; inputting the concentration data to a decision tree ensemble configured to predict an odor intensity of the aqueous polymer composition after polymerisation based on the concentration data; and outputting a predicted odor intensity of the aqueous polymer composition from the decision tree ensemble.

### FIELD

The present invention relates to a method and a system for odor prediction for an aqueous polymer composition and a coating made therefrom by machine learning, particularly suitable for coating applications.

### INTRODUCTION

Aqueous or waterborne binder or coating compositions are becoming increasingly more important as environmentally friendly alternatives to solvent-based compositions. In the architectural coating industry, especially for interior applications, some manufacturers and end users are also keen on odor of aqueous compositions. Conventional odor evaluation of binders and coating compositions mostly depends on human sensory panels. Such odor panel testing is laborious and time consuming, which also tends to be subjective and usually needs to be repeated in order to get consistent test results. In addition, long-time exposure to odor may cause potential hazards to odor panelists. Existing computer-implemented odor prediction approaches are typically developed for relatively simple systems such as tobacco, which are not applicable to aqueous polymer compositions or coatings in the coating industry which have more complex chemical compositions. These aqueous polymer compositions typically comprise various types (e.g., more than 30 types) of volatile organic compounds at concentrations in a wide range (e.g., from part per billion to part per million of the aqueous polymer composition) and the interactions between these compounds usually have significant impacts on the odor of the aqueous polymer compositions. Therefore, it is more challenging to develop a method or system for odor prediction of aqueous polymer systems.

It is therefore desirable to provide a method of prediction of odor of an aqueous polymer composition or a coating made therefrom.

### SUMMARY

The present invention provides a novel computer-aided method and system without the aforementioned problems. The method of the present invention includes inputting novel combinations and concentrations of volatile organic compounds (VOCs) in aqueous polymer compositions or coatings made therefrom to a specific supervised machine learning module to predict the odor intensity of the composition or coating. The machine learning module used in the present invention is a decision tree ensemble that is trained to predict the odor intensity of aqueous polymer compositions or coatings using a training dataset using a plurality of training samples. The training dataset comprises the concentration data and the corresponding actual odor intensity rated by human panelists for aqueous polymer compositions or coatings containing VOCs. Such method or system enables a standardized and automated approach to accurately predict the odor through rating the odor intensity. As compared to odor evaluation by human panelists, the method of the present invention greatly improves the test consistency and accuracy without impacts from variances of panelists, experimental conditions, and environmental conditions. At the same time, the method can also greatly improve productivity and efficiency for quickly evaluating dozens of samples in one sequence, thus reducing labor costs and time on training and evaluation and also preventing panelists exposure to hazardous substances by inhalation.

In a first aspect, the present invention is a method of predicting odor of an aqueous polymer composition.

The method comprises:
analytically characterizing the aqueous polymer composition with a detector, thereby generating concentration data for volatile organic compounds in the aqueous polymer composition from the analytical characterization;
inputting the concentration data to a decision tree ensemble configured to predict an odor intensity of the aqueous polymer composition based on the concentration data; and
outputting a predicted odor intensity of the aqueous polymer composition from the decision tree ensemble.

In a second aspect, the present invention is a method of predicting odor of a coating. The method comprises:
analytically characterizing the coating with a detector, thereby generating concentration data for volatile organic compounds in the coating, wherein the coating is obtained by drying an aqueous polymer composition;
inputting the concentration data to a decision tree ensemble configured to predict an odor intensity of the coating based on the concentration data; and
outputting a predicted odor intensity of the coating from the decision tree ensemble.

In a third aspect, the present invention is a system for predicting odor of an aqueous polymer composition or a coating made therefrom. The system comprises:
a detector, configured to analytically characterize the aqueous polymer composition of the coating, thereby generating concentration data for volatile organic compounds from the analytical characterization; and a computing device with a decision tree ensemble deployed thereon, configured to input the concentration data and output a predicted odor intensity of the aqueous polymer composition or the coating.

### BRIEF DESCRIPTION OF THE DRAWINGS

FIG. 1 illustrates a schematic of a simplified example of a decision tree for prediction.

FIG. 2 illustrates a flow chart of an odor prediction method in accordance with one example of the present invention.

FIG. 3 illustrates a schematic drawing of a cloud-based server cluster in accordance with one example of the present invention.

FIG. 4 illustrates a schematic block diagram of an odor prediction system in accordance with one example of the present invention.

### DETAILED DESCRIPTION

Test methods refer to the most recent test method as of the priority date of this document when a date is not indicated with the test method number.

Products identified by their tradename refer to the compositions available under those tradenames on the priority date of this document.

“And/or” means “and, or as an alternative”. All ranges include endpoints unless otherwise indicated.

“Odor” refers to the sensation perceived through the nose by the olfactory nerves.

“Odor intensity” is a measure of how strong an odor is based on an initial perception. The odor intensity can be rated based on criteria described in the VDA 270 standard. The VDA 270 standard (Determination of Odor Characteristics of Trim Materials in Motor Vehicles) is developed by the German Automotive Industry Association (VDA).

“Actual odor intensity” is the odor intensity rated by human panelists according to the Odor Panel Testing described in the Examples section below. “Volatile organic compound” (“VOC”) refers to any organic compound with a normal boiling point of 250 degrees Celsius (° C.) or lower at a pressure of 101.3 kilopascals (kPa).

“VOC profile” means a dataset comprising identification of VOCs and their concentrations.

“Aqueous” polymer composition herein means a composition comprising a polymer present in an aqueous medium, e.g., polymer particles dispersed in an aqueous medium. By “aqueous medium” herein is meant water and from 0 to 30%, by weight based on the weight of the medium, of water-miscible compound(s) such as, for example, alcohols, glycols, glycol ethers, glycol esters, or mixtures thereof.

“Acrylic (co)polymer” herein refers to a homopolymer of an acrylic monomer or a copolymer comprising structural units of an acrylic monomer with one or more additional monomers. “Acrylic” in the present invention includes (meth)acrylic acid, (meth)alkyl acrylate, (meth)acrylamide, (meth)acrylonitrile and their modified forms such as (meth)hydroxyalkyl acrylate. Throughout this document, the word fragment “(meth)acryl” refers to both “methacryl” and “acryl”. For example, (meth)acrylic acid refers to both methacrylic acid and acrylic acid, and methyl (meth)acrylate refers to both methyl methacrylate and methyl acrylate. Specific examples of acrylic (co)polymer include acrylic homopolymers, styrene acrylic copolymers, or mixtures thereof.

“Structural units”, also known as “polymerized units”, of the named monomer, refers to the remnant of the monomer after polymerization, that is, polymerized monomer or the monomer in polymerized form. For example, a structural unit of methyl methacrylate is as illustrated:

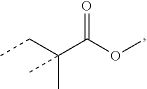

where the dotted lines represent the points of attachment of the structural unit to the polymer backbone.

“Machine learning” refers to a set of methods that ‘learn’ from data to improve performance on specific tasks. Machine learning algorithms build models based on historical data, also known as training data to make predictions as the model outputs.

“Decision tree” is a type of machine learning in which a model has a tree-like structure to represent the ‘tests’ on a series of attributes. Each internal node represents the ‘test’, and each leaf node represents the decisions following the outcomes of the attributes.

The method of the present invention is useful for predicting the odor of an aqueous polymer composition or a coating made therefrom (collectively as “test sample”). Particularly, the method is useful for predicting the odor of the aqueous polymer composition.

The method of the present invention comprises analytically characterizing the aqueous polymer composition or the coating with a detector, thereby generating the concentration data for volatile organic compounds in the aqueous polymer composition or the coating.

Aqueous polymer compositions, particularly, in coating applications, typically comprise various types of VOCs, for example, more than twenty types of VOCs. These VOCs may be present at a wide range of concentrations, from part per billion to part per million of the compositions. VOC types and interactions between these VOCs usually have significant impact on the perceived odor of aqueous polymer compositions. Thus, odor intensity from a mixture of individual chemicals cannot reflect the actual odor of aqueous polymer compositions comprising these individual chemicals. By using the concentration data for a mixture of VOCs in aqueous compositions, that is obtained from the analytical characterization, for training the specific machine learning module, the present invention can achieve surprisingly higher prediction accuracy than modules which generate a predicted odor intensity by inputting individual chemical structure data (such as chemical molecule descriptors) for training, or by measuring individual chemicals for training.

### Aqueous Polymer Composition

The aqueous polymer composition useful in the present invention may comprise one or more acrylic (co)polymers. The acrylic (co)polymers may comprise any one or any combination of more than one type of an alkyl ester of (meth)acrylic acid. “Alkyl” means a linear, branched, or cyclic alkyl group. Alkyl esters of (meth)acrylic acid can be C₁-C₂₀-, C₁-C₁₀-, or C₁-C₈-alkyl esters of (meth)acrylic acid including, for example, methyl acrylate, methyl methacrylate, ethyl acrylate, butyl acrylate, butyl methacrylate, 2-ethylhexyl acrylate, iso-butyl (meth)acrylate, hexyl (meth)acrylate, lauryl (meth)acrylate, stearyl (meth)acrylate, cyclohexyl (meth)acrylate, benzyl (meth)acrylate, oleyl (meth)acrylate, palmityl (meth)acrylate, nonyl (meth)acrylate, decyl (meth)acrylate, dodecyl (meth)acrylate, pentadecyl (meth)acrylate, hexadecyl (meth)acrylate, octadecyl (meth)acrylate, or mixtures thereof. Desirably, the alkyl esters of (meth)acrylic acid comprise methyl methacrylate, methacrylate, ethyl acrylate, butyl methacrylate, butyl acrylate, 2-ethylhexyl acrylate, or mixtures thereof. The acrylic (co)polymer may comprise structural units of the alkyl ester of (meth)acrylic acid at a concentration of 5% to 100%, and can be 15% to 99%, 30% to 98%, 50% to 95%, 55% to 90%, or 60% to 90%, by weight based on the weight of the acrylic (co)polymer.

The acrylic (co)polymer useful in the present invention may comprise or be free of structural units of one or more ethylenically unsaturated monomers carrying at least one functional group selected from an amide, ureido, carbonyl, carboxyl, carboxylic anhydride, hydroxyl, sulfonic acid, sulfonate, phosphoric acid, or phosphate group; or salts thereof (hereinafter “functional monomer”). Suitable functional monomers may include α, β-ethylenically unsaturated carboxylic acids including an acid-bearing monomer such as methacrylic acid, acrylic acid, itaconic acid, maleic acid, or fumaric acid; or a monomer bearing an acid-forming group which yields or is subsequently convertible to, such an acid group (such as anhydride, (meth)acrylic anhydride, or maleic anhydride); phosphorous containing monomers such as vinyl phosphonic acid, allyl phosphonic acid, phosphoalkyl (meth)acrylates such as phosphoethyl (meth)acrylate, phosphopropyl (meth)acrylate, phosphobutyl (meth)acrylate, salts thereof, or mixtures thereof; sulfonic acid monomers and salts thereof including, for example, 2-acrylamido-2-methyl-1-propanesulfonic acid; sodium salt of 2-acrylamido-2-methyl-1-propanesulfonic acid; and ammonium salt of 2-acrylamido-2-methyl-1-propane sulfonic acid; sodium vinyl sulfonate; sodium salt of allyl ether sulfonate; monomers bearing carbonyl-containing groups such as acetoacetoxyethyl methacrylate (AAEM) and diacetone acrylamide (DAAM); acrylamide; methacrylamide; or mixtures thereof. Desirably, the functional monomer comprises acrylamide, acrylic acid, methacrylic acid, phosphoethyl (meth)acrylate, sodium salt of 2-acrylamido-2-methyl-1-propanesulfonic acid, or mixtures thereof. The acrylic (co)polymer may comprise structural units of the functional monomer at a concentration of zero to 10%, and can be 0.1% to 8%, 0.3% to 5%, or 0.5% to 3%, or 0.7% to 2%, by weight based on the weight of the acrylic (co)polymer.

The acrylic (co)polymer useful in the present invention may comprise or be free of structural units of one or more additional ethylenically unsaturated nonionic monomers other than the alkyl ester of (meth)acrylic acid and the functional monomer described above. The term “nonionic monomers” refers to monomers that do not bear an ionic charge between pH=1-14. Suitable additional ethylenically unsaturated nonionic monomers may include vinyl aromatic monomers such as styrene and substituted styrene (including for example .alpha.-methyl styrene, p-methyl styrene, t-butyl styrene, vinyltoluene), glycidyl (meth)acrylate, ethylenically unsaturated monomers carrying at least one alkoxysilane functionality including vinyltrialkoxysilanes such as vinyltrimethoxysilane and (meth)acryloxyalkyltrialkoxysilanes such as (meth)acryloxyethyltrimethoxysilane and (meth)acryloxypropyltrimethoxysilane; α-olefins such as ethylene, propylene, and 1-decene; vinyl acetate vinyl acetate, vinyl butyrate, vinyl versatate and other vinyl esters; acrylonitrile; or mixtures thereof. Desirably, the additional ethylenically unsaturated nonionic monomer comprises styrene, vinyl acetate, or mixtures thereof. The acrylic (co)polymer may comprise or be free of structural units of a multifunctional nonionic monomer such as butadiene, divinylbenzene, and allyl (meth)acrylate, typically at a concentration of zero to 5%, and can be zero to 2%, 0.1% to 1%, or 0.1% to 0.5%, by weight based on the weight of the acrylic (co)polymer.

The acrylic (co)polymer useful in the present invention may comprise structural units of the additional ethylenically unsaturated nonionic monomer at a concentration of zero to 95%, and can be 1% to 85%, 2% to 70%, 5% to 50%, or 10% to 45%, by weight based on the weight of the acrylic (co)polymer. Desirably, the acrylic (co)polymer may comprise structural units of styrene at a concentration of zero to 60%, and can be 5% to 55%, 10% to 50%, or 20% to 40%, by weight based on the weight of the acrylic (co)polymer. Alternatively, the acrylic (co)polymer may comprise structural units of vinyl acetate at a concentration of zero to 95%, and can be 5% to 90%, 10% to 85%, 20% to 80%, or 50% to 70% by weight based on the weight of the acrylic (co)polymer.

The acrylic (co)polymer useful in the present invention can be a vinyl acrylic (co)polymer comprising 5% to 30% of structural units of the alkyl ester of (meth)acrylic acid, 70% to 95% of structural units of vinyl acetate, and zero to 5% of structural units of the functional monomer. Alternatively, the acrylic (co)polymer is a styrene acrylic copolymer comprising 30% to 60% of structural units of styrene, 40% to 70% of structural units of the alkyl ester of (meth)acrylic acid, and zero to 5% of structural units of the functional monomer. Alternatively, the acrylic (co)polymer may comprise 95% to 100% of structural nits of the alkyl ester of (meth)acrylic acid and zero to 5% of structural units of the functional monomer.

The aqueous polymer composition useful in the present invention may comprise one or more types of VOCs. Depending on the synthesis process such as polymerization process for preparing the aqueous polymer composition, VOCs in the aqueous polymer composition may be selected from aldehydes, ketones, acrylic monomers, alcohols, saturated esters, ethers, aromatic hydrocarbons, or mixtures thereof.

The aqueous polymer composition can be a binder emulsion or an aqueous coating composition. Commercially available aqueous polymer compositions include, for example, PRIMAL™ DC-420 emulsion, PRIMAL™ DC-430V emulsion, PRIMAL™ SF-155 emulsion, PRIMAL™ DC-460 emulsion, PRIMAL™ DC-480 emulsion, PRIMAL™ LE-328V emulsion, PRIMAL™ AS-2010 emulsion, PRIMAL™ AS-356 emulsion, PRIMAL™ SF-508 emulsion, PRIMAL™ SF-105 emulsion, PRIMAL™ SF-308 emulsion, PRIMAL™ LE-318V emulsion, or mixtures thereof (PRIMAL is a trademark of The Dow Chemical Company).

The aqueous polymer composition useful in the present invention may comprise or be free of a pigment, an extender or mixtures thereof. Pigments may include particulate inorganic materials which are capable of materially contributing to the opacity or hiding capability of a coating. Such materials typically have a refractive index greater than 1.8. Examples of suitable pigments include titanium dioxide (TiO₂), zinc oxide, zinc sulfide, iron oxide, barium sulfate, barium carbonate, or mixtures thereof. The aqueous polymer composition may comprise or be free of one or more extenders. Extenders may include particulate inorganic materials typically having a refractive index of less than or equal to 1.8 and greater than 1.5. Examples of suitable extenders include calcium carbonate, aluminum oxide (Al₂O₃), clay, calcium sulfate, aluminosilicate, silicate, zeolite, mica, diatomaceous earth, solid or hollow glass, ceramic bead, and opaque polymers such as ROPAQUE™ Ultra E opaque polymer available from The Dow Chemical Company (ROPAQUE is a trademark of The Dow Chemical Company), or mixtures thereof. The pigment and/or extender may be present, by weight based on the weight of the aqueous polymer composition, at a concentration of from zero to 40%, from 5% to 30%, from 10% to 25%, or from 15% to 20%. The aqueous polymer composition may further comprise or be free of any one or combination of the following additives that are commonly used in the coating applications: defoamers, thickeners, dispersants, biocides, and coalescents.

### Analytical Characterization

The method of the present invention comprises analytically characterizing the aqueous polymer composition with a detector, hereby generating the concentration data for VOCs in the aqueous polymer composition. “Concentration data for VOCs”, also as “VOC concentration data”, refers to a plurality of concentration values of VOCs in the aqueous polymer composition. Alternatively, the concentration data can include the concentration values of those VOCs that each has a concentration of 0.1 part per million (ppm) or more, 0.2 ppm or more, 0.3 ppm or more, or even 0.5 ppm or more, of the weight of the aqueous polymer composition. The concentration of a VOC refers to the weight concentration of the VOC in ppm, based on the weight of the aqueous polymer composition.

Surprisingly, it is also discovered that the concentration of some specific VOCs such as compounds having one to 11 carbon atoms and a normal boiling point less than 220° C. at a pressure of 101.3 kPa play a vital role in the prediction accuracy of the odor intensity of such aqueous polymer compositions. Desirably, the concentration data that is input to the decision tree ensemble comprises the concentrations of VOCs selected from acetone, 2-methyl propanol, 1-butanol, methyl methacrylate, butyl acetate, 4-heptanone, 2-heptanone, butyl ether, styrene, butyl acrylate, anisole, propanoic acid, butyl ester, methyl ethyl benzene, 3-methyl-4-heptanone, propenyl benzene, propyl benzene, benzaldehyde, acetophenone, butyl methacrylate, isobutyl vinylacetate, butanoic acid, butyl ester, 2-butenoic acid, butyl ester, diethyl benzene isomers, cyclohexyl methacrylate (2-propenoic acid, 2-methyl-, cyclohexyl ester), 2-ethylhexyl acrylate, xylene, ethyl benzene, or mixtures thereof. More desirably, the concentration data comprises or consists of the concentrations of VOCs selected from 2-methyl propanol, methyl methacrylate, butyl acetate, 2-heptanone, 3-methyl-4-heptanone, styrene, butyl acrylate, propanoic acid, butyl ester, 2-ethylhexyl acrylate, ethyl benzene, xylene, benzaldehyde, or mixtures thereof.

Analytically characterizing the aqueous polymer composition may comprise analytical characterization techniques that have a lower limit of detection (LLOD)<0.1 ppm. LLOD (also known as analytic sensitivity) is the smallest amount of an analyte that can reliably be detected. Typical analytical characterization techniques can be gas chromatography (GC) coupled with different detectors, such as GC-MSD (Mass Selective Detector) (which is used interchangeably with “GC-MS” below), GC-FID (Flame Ionization Detector), and GC-ECD (Electron Capture Detector). GC-MS typically comprises a GC instrument and an MSD. The detector in GC-MS can be used to identify VOCs by the MS spectra of the VOCs, and also can be used to obtain the peak areas of the VOCs for further quantification of their concentrations. By comparing the peak area of the VOCs in the aqueous polymer composition with peak area of external standards, the concentration of each VOC can be obtained. Conventional GC-MS can be used, such as an Agilent 6890 gas chromatograph coupled with Agilent 5975C MSD. Various sample preparation techniques for GC-MS can be used, including, for example, solid phase micro-extraction (SPME) coupled with GC-MS (also as “SPME GC-MS”), needle trap microextraction (NTM) coupled with GC-MS, and Tenax absorbent cartridge (TC) coupled with GC-MS. Conventional NTM GC-MS and TC GC-MS are available from Shinwa Ltd. (Japan) and Gerstel Co., Ltd. (Germany), respectively.

Desirably, SPME coupled with GC-MS comprising a SPME coupled to a GC equipped with a MSD instrument is used to extract, identify, and quantify VOCs from the aqueous polymer composition. SPME is a sample preparation technique for integrating operations including sample collection, extraction, and analyte enrichment from the headspace of a sample. The SPME technique in the present invention is coupled to GC and can be used to extract analytes (such as VOCs) from liquid samples such as the aqueous polymer composition. A typical procedure for SPME comprises two steps: (1) partitioning of analytes between the extraction phase and the sample matrix, and (2) subsequent desorption of concentrated extracts into an analytical instrument, such as GC-MS. The SPME procedure can be performed manually or automatically. For example, a multi-purpose sampler (MPS) can be used to automate the SPME procedure.

### Decision Tree Ensemble

The VOC concentration data generated from the analytical characterization is used as the input to a decision tree ensemble configured to predict the odor intensity of the aqueous polymer composition based on the VOC concentration data, for example, a decision tree ensemble is trained to predict the odor intensity.

A decision tree is a model that uses a tree-like structure of decisions and their possible consequences, such as the chance of event outcomes or predicted values. The decision tree model, an example of a supervised learning model, is trained using input data paired with output data by optimizing the model parameters to minimize the difference between the model predicted values and the actual values.

Then, the trained model can predict the new output given the set of input data from a test sample (also “a new sample”). “Decision tree ensemble” (also known as “decision tree ensemble model”) refers to a model that combines multiple decision trees. “Multiple” means two or more, and can be 10 or more, while at the same time is generally 1000 or less or 100 or less.

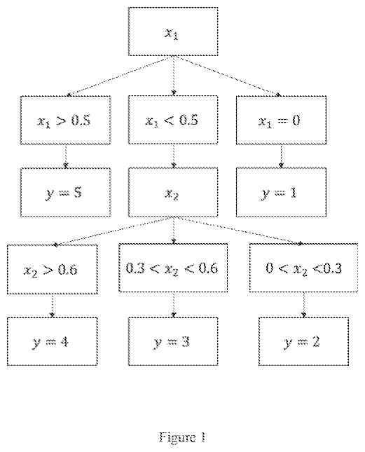

FIG. 1 illustrates a schematic of a simplified decision tree example with a decision-making process of prediction of y based on p features as input X={x₁, x₂ . . . xₚ}. For example, in a decision tree using two features, x₁ and x₂, each node is split into different branches based on the condition of x₁ or x₂. Based on the condition of x₁, the decision tree is first split into 3 branches of different ranges of x₁. When x₁>0.5, the decision tree output is y=5. When x₁<0.5, there is another branch splitting based on the condition of x₂. In these new branches, the predicted value of y can be 2, 3, or 4 depending on the different conditions of the values of x₂ under the condition of x₁<0.5, for example, when x₁<0.5 and x₂>0.6, the predicted y is 4.

A decision tree can be built for regression by using Standard Deviation Reduction for making leaf nodes. In a decision tree regression task (where the predicted y is a continuous numerical value), a decision tree is built top-down from a root node to leaf nodes and involves partitioning data that contain similar values by standard deviation. A decision tree has two or more branches, and each branch represents the values for attribute tested. Each leaf node represents a decision on the target. The count m stands for the total number of points in the node, \tilde{z} calculates the average values of the attribute Z={z₁, z₂ . . . zₘ} as below:

$$\overline{z} = \frac{{\sum }_{i = 1}^{m} {z}_{i}}{m}$$

The standard deviation(S) is for tree branching:

$$S = \sqrt{\frac{{\sum }_{i = 1}^{m} {( {z}_{i} - \overline{z} )}^{2}}{m}}$$

The coefficient of variation (CV) is used for stopping the branching:

$$CV = \frac{S}{\overline{z}} \times 100 \%$$

The sum of standard deviation (S(target, C)) of multiple attributes can be computed from the combination of the standard deviation of the individual standard deviation (S) and the probability (P) of the condition (c) happening under all conditions of the decision (C):

$$S ( target , C ) = {\sum }_{c ∈ C} P ( c ) S ( c )$$

The standard deviation reduction (SDR) can be achieved after the dataset is split on a feature. The decision tree is built to seek the highest SDR, which is calculated by the difference between the standard deviation of the node before the branching (S(target)) and after the branching (S(target, C)):

$$SDR ( target , C ) = S ( target ) - S ( target , C )$$

The branching is stopped after the CV reaches to the designated threshold. Sometimes the branching is also limited by other criteria including maximum depth of tree and minimum samples per node.

A decision tree ensemble method combines several decision trees to produce better prediction performance than using a single tree. The decision tree ensemble used in the present invention is trained to predict the odor intensity of a sample by building a correlation model from the VOC concentrations to the odor intensity rated by human sensory panelists (hereinafter also referred as “actual odor intensity”) in a training phase. During the training phase, the concentrations of VOCs in training samples serve as the inputs to the decision tree ensemble model, and the actual odor intensity of these training samples that are rated by panelists serve to guide the learning of the output to minimize the difference between the actual and predicted values. The trained decision tree ensemble model can be used to predict the odor intensity based on the VOC concentrations of a new sample.

First, each of the concentrations among selected VOCs for the total p features are collected: {P₁, P₂ . . . Pₚ}, where each feature group P contains n samples: P={p₁, p₂ . . . pₙ} that are rescaled so that all values fall on the interval [0,1]:

$${p}_{i} = \frac{{p}_{i} - min ( P )}{max ( P ) - min ( P )}$$

where max(P) is the maximum concentration of a VOC among all training samples and min(P) is the minimum concentration of such VOC among all training samples.

The decision tree ensemble model takes the rescaled inputs from the training data to predict the odor intensity as the outputs. Parameters for the model are optimized by minimizing the mean squared error (MSE) between the actual value y; and the predicted value ŷᵢ, as shown in equation (I) below:

$$MSE = \frac{1}{n} {\sum }_{i = 1}^{n} {( {y}_{i} - {\hat{y}}_{i} )}^{2} \qquad ( I )$$

where n is the total number of data points, yᵢ is the actual value of the odor intensity rated by human panelists, i is the sample data point, and ŷᵢ is the model predicted value of the odor intensity.

MSE is a metric used to measure the average of the squares of the difference between the predicted values and the actual values. RMSE means root mean squared error that is a metric to measure the difference between the predicted values and the actual values. Percentage RMSE is the square root of MSE normalized by the population mean to a dimensionless number expressed as a percentage. The percentage RMSE can be calculated as below:

$$percentage RMSE = \frac{{\sum }_{i}^{n} \frac{{( \hat{{y}_{l}} - {y}_{i} )}^{2}}{n}}{\overline{{y}_{i}}} \qquad ( II )$$

where n, yᵢ, i and ŷᵢ are as defined above in equation (I) above, and y̅ᵢ̅ is the mean value for the actual odor intensity rated by human panelists. The lower the percentage RMSE, the better the model fits a dataset.
R² means the coefficient of discrimination, which is a commonly used performance metric for regression that calculates the proportion of variance explained by a regression model. R² normally ranges from 0 to 1 and can be calculated according to equation (III) below:

$${R}^{2} = 1 - \frac{{\sum }_{i} {( {y}_{i} - {\hat{y}}_{l} )}^{2}}{{\sum }_{i} {( {y}_{l} - \overline{{y}_{i}} )}^{2}} \qquad ( III )$$

where yᵢ, i and ŷᵢ are as defined above in equation (I) above, and y̅ᵢ̅ is the mean value for the actual odor intensity rated by human panelists. The higher the R², the better the model fits a dataset.

Multiple decision-tree-based ensemble learning models can be used in the present invention, including, for example, Random Forest (RF) model, Gradient Boosting (GB) model, and extreme Gradient Boosting (XGB) model.

The RF model uses over bagging, which creates several subsets of the data from the training sample, while performing a random selection of training features to develop numerous decision trees with the selected samples and features. Averaging the decision trees results in the final RF model. The RF model can effectively reduce the variance of a single decision tree.

The GB model uses boosting, which is another ensemble technique to combine multiple weak learners to make a strong one. In the GB model, each individual decision tree learns sequentially, where early learners learn simple models to the data, and the later learners analyzes the prediction errors by learning the errors from the earlier learners. It uses a gradient descent algorithm, which allows a differentiable loss function to optimize weights of the sequential learners to recover the difference between the actual and predicted values.

The XGB model provides several adjustments on the GB algorithm, including changing the gradient descent algorithm in the GB algorithm to a Newton-Raphson optimization algorithm. The XGB model also uses extra randomization parameters, proportional shrink in the leaf nodes, penalization of trees, and feature selection in addition to the original GB algorithm.

The RF model, GB model and XGB model can be built in a computational environment, such as Python, R, or MATLAB. These models are available in open-source libraries such as the Scikit-learn library. For example, the RF and GB models can be built with a Scikit-learn package as described in “Scikit-learn: Machine Learning in Python”, Pedregosa, F. et al., Journal of Machine Learning Research, 2011, 12, 2825-2830. The XGB model can be built with an XGBoost Package as described in “XGBoost: A Scalable Tree Boosting System”, Chen, T., and Guestrin, C., Proceedings of the 22^{nd} ACM SIGKDD International Conference on Knowledge Discovery and Data Mining, 2016, 785-794, Association for computing machinery (ACM), New York, USA.

Hyperparameters for the decision tree ensemble models can be default settings in a machine learning model library such as the Scikit-learn library. “Hyperparameters” are defined values used to control the learning process that are set before the training process of a model begins. Hyperparameters are different from other parameters of the model because other parameters are learned during the training process. Examples of hyperparameters include number of estimators which stands for how many trees are used for the ensemble learning model, and minimum number of samples per split which stands for the criteria for making a split in the decision tree model.

The decision tree ensemble useful in the present invention is trained (i.e., trained decision tree ensemble) to predict the odor intensity of the aqueous polymer composition using a training dataset using a plurality of training samples, such as the concentration data for aqueous compositions containing VOCs. The training dataset comprises the VOC concentration data (as the input) for each training sample paired with the corresponding actual odor intensity data (as the output) rated by human panelists for such training sample, thereby giving a trained decision tree ensemble. The obtained training results can be validated with a validation dataset using validation samples to monitor the model prediction accuracy. The decision tree ensemble model may be further tested with a test dataset using another set of test samples which are prepared separately at a different time from the samples used for generating the training dataset. Models are selected that meet certain thresholds of training performance, validation accuracy, and testing accuracy.

In the present invention, concentrations of selected VOCs in each training sample, such as the aqueous polymer composition described above or a coating described below, are input to a decision tree ensemble model to train the model. Desirably, the training dataset comprises the concentration data of volatile organic compounds that each has a concentration ≥0.1 ppm, by weight based on the weight of the training samples (i.e., training aqueous polymer compositions). More desirably, the training dataset comprises the concentration data of volatile organic compounds comprising acetone, 2-methyl propanol, 1-butanol, methyl methacrylate, butyl acetate, 4-heptanone, 2-heptanone, butyl ether, styrene, butyl acrylate, anisole, propanoic acid, butyl ester, methyl ethyl benzene, 3-methyl-4-heptanone, propenyl benzene, propyl benzene, benzaldehyde, acetophenone, butyl methacrylate, isobutyl vinylacetate, butanoic acid, butyl ester, 2-butenoic acid, butyl ester, diethyl benzene or isomers, cyclohexyl methacrylate, 2-ethylhexyl acrylate, xylene, ethyl benzene, or mixtures thereof. The training samples are analytically characterized to determine the concentrations of VOCs using methods such as GC-MS (as the input) and evaluated for odor intensity from human sensory results (i.e., ratings by human panelists), also as “actual odor intensity”. The actual odor intensity of the training samples can be evaluated by odor panel testing according to the VDA 270 standard on a scale of 1 to 6, where 1=Not perceptible; 2=Perceptible, not disturbing; 3=Clearly perceptible, but not disturbing; 4=Disturbing; 5=Strongly disturbing; and 6=Not acceptable (further details provided in the Examples section below). The decision tree ensemble models are trained to model the correlation between the VOC concentration data and the odor intensity of the aqueous polymer composition rated by panelists. The decision tree ensemble models in the present invention can be trained to afford accuracy as indicated by training R²>0.85, as calculated by the equation (III) above. The trained decision tree ensemble models can be further validated using a validation dataset using a plurality of validation samples. The validation dataset comprises VOC concentration data (as the input) for volatile organic compounds in each validation sample and the corresponding actual odor intensity data (as the output) rated by human panelists for such validation sample.

The trained decision tree ensemble model can then predict the odor of an aqueous polymer composition to be tested as a test sample (also as “new sample”) using a test dataset comprising the concentration data of VOCs in the test sample obtained from the analytical characterization. Users can input the concentration data of VOCs for the test sample to the trained decision tree ensemble via a web-based user interface (details described below).

In one embodiment of the present invention, a decision tree ensemble model is trained using a total of 39 pairs of data comprising actual odor intensity rated by human panelists, which is split into a training dataset with 31 data pairs and a validation dataset with 8 data pairs. “Data pair” herein refers to VOC concentration data for a particular sample and its corresponding odor intensity as rated by panelists. Then the model is evaluated or tested with a new dataset collected in a new batch of experiments for evaluating the prediction performance of the model.

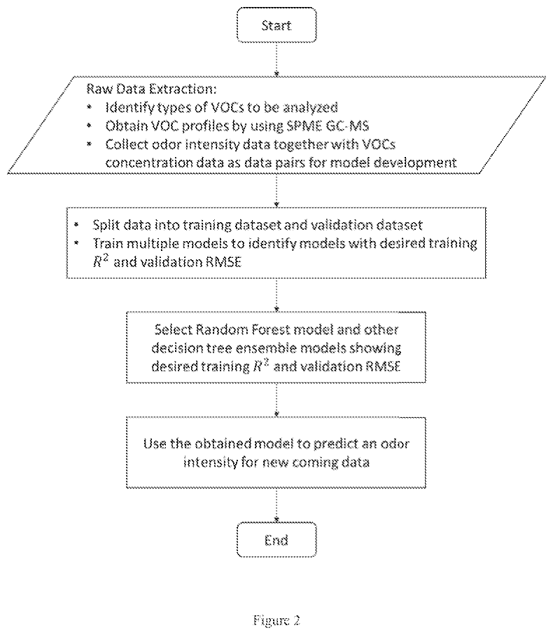

FIG. 2 illustrates a flow chart of an odor prediction method in accordance with one example of the present invention. The method includes raw data extraction by identifying types of VOCs to be analyzed, obtaining VOC profiles containing the concentration data for VOCs, and collecting the odor intensity data from the odor panel testing (further details provided in the Examples section below). The odor intensity data together with the concentration data as data pairs are used for model development. The method further comprises training and validating multiple models with the obtained concentration data paired with the odor intensity data, and then selecting an appropriate model based on desired model criteria including training R² and validation RMSE. Underfitting is a scenario where a model cannot capture the relationship between the input and output variables. The underfitted models tend to have undesirably low training R². The training R² threshold, i.e., training R²>0.85, is used to filter out underfitted models. Nevertheless, overfitting of a model is when the model is too closely aligned to the training data, and the learned representation cannot predict the validation data accurately. The validation percentage RMSE threshold, i.e., validation percentage RMSE<30%, is used to filter out overfitted models. After satisfying the thresholds, the trained models are used to predict the odor intensity of new samples.

The method of the present invention may further comprise adjusting the synthesis process of an aqueous polymer composition based on the predicted odor intensity, such as adjusting the polymerization process, particularly, emulsion polymerization process, for preparing the polymer in the aqueous polymer composition. Parameters that can be adjusted may include, for example, surfactant types and amounts, initiator types and amounts, monomer types and sources, reaction temperatures, steam stripping parameters, or combinations thereof.

### Coating

The present invention also relates to a method of predicting the odor of a coating (also as “coating film”). The method comprises analytically characterizing the coating with a detector, thereby generating the concentration data for VOCs in the coating, inputting the concentration data to a decision tree ensemble configured to predict an odor intensity of the coating based on the VOC concentration data; and outputting a predicted odor intensity of the coating from the decision tree ensemble. The coating can be obtained by drying, or allowing to dry, the aqueous polymer composition described above, i.e., dried aqueous polymer composition. The aqueous polymer composition can be applied to a substrate, and drying, or allowing to dry, the applied polymer composition to form the coating. The coating may have a dry film thickness of 50 to 60 μm. The aqueous polymer composition can be used alone, or in combination with other coatings to form multi-layer coatings. The aqueous polymer composition can be applied to the substrate by incumbent means including brushing, dipping, rolling, and spraying. Drying can be conducted at room temperature (20° C. to 25° C.), or at an elevated temperature, for example, from 35° C. to 60° C. As a result of drying, the coating may comprise different VOC profiles from the aqueous polymer composition above, as some of the VOCs in the aqueous polymer composition may evaporate from the composition after drying. The coating is suitable for different coating applications, such as architectural coatings, wood coatings and protective coatings. The analytical characterization useful for generating the VOC concentration data for the coating is the same as described above for analytically characterizing the aqueous polymer composition. The decision tree ensemble used in the method of prediction odor of the coating is as described in the method of prediction odor for the aqueous polymer composition above, except that the training samples, validation samples and test samples used in the decision tree ensemble are coating samples that are obtained by drying the aqueous polymer composition.

Particularly, the novel combination of the VOC concentration data as the input with a specific machine learning model, that is, the decision tree ensemble, enables the method of the present invention predict the odor intensity of the aqueous polymer composition with a higher accuracy than other machine learning models such as an Artificial Neural Network (ANN) model, Partial Least Square Regression (PLS) model, or Ridge Regression (RR) model. The ANN model is based on a collection of connected units or nodes called artificial neurons for predictive modeling. The PLS model finds a linear regression model by projecting the predicted variables and the observable variables to a new space. The RR model builds multiple-regression models in scenarios where independent variables are highly correlated by creating a ridge regression estimator.

In one embodiment, the method can provide high prediction accuracy for predicting the odor of the aqueous polymer composition, as indicated by training R²>0.85 as calculated according to the equation (III) above, and test percentage RMSE less than 30% (<30%) as calculated according to the equation (II) above. In addition, validation percentage RMSE is <30% as calculated according to the equation (II) above. “Training R²” refers to R² for the training dataset, “validation percentage RMSE” refers to the percentage RMSE for the validation dataset, and “test percentage RMSE” refers to the percentage RMSE for the test dataset. Particularly, the method when using the RF model can provide even higher prediction accuracy, showing test percentage RMSE less than 20%.

### System for Odor Prediction

The present invention also relates to a system for predicting the odor of the aqueous polymer composition or the coating. The system of the present invention comprises: a detector configured to analytically characterize the aqueous polymer composition or the coating, thereby generating concentration data for volatile organic compounds in the aqueous polymer composition or in the coating from the analytical characterization; and a computing device with a decision tree ensemble deployed thereon, configured to input the concentration data and output a predicted odor intensity. The decision tree ensemble and training the decision tree ensemble are as described above. For example, the decision tree ensemble deployed on the computing device is trained using a training dataset using a plurality of training samples, where the training dataset, the training samples, and the resulting trained decision tree ensemble are as described above. Analytically characterizing and the detector are as described above, such as GC-MS, and desirably, SPME GC-MS.

The computing device useful in the present invention may comprise a processor and data storage, where the data storage has stored thereon computer-executable instructions that, when executed by the processor, cause the computing device to carry out functions comprising inputting the VOC concentration data to the decision tree ensemble and outputting the predicted odor intensity.

The computing device useful in the present invention can be a client device (e.g., a device actively operated by a user), a server device (e.g., a device that provides computational services to client devices), or some other type of computational platform. Some server devices can operate as client devices from time to time in order to perform particular operations, and some client devices can incorporate server features.

The processor useful in the present invention can be one or more of any type of computer processing element, such as a central processing unit (CPU), a co-processor (e.g., a mathematics, graphics, neural network, or encryption co-processor), a digital signal processor (DSP), an application specific integrated circuit (ASIC), a network processor, and/or a form of integrated circuit or controller that performs processor operations.

The data storage can include one or more data storage arrays that include one or more drive array controllers configured to manage read and write access to groups of hard disk drives and/or solid-state drives.

In some embodiments, the computing device can be deployed to support a clustered architecture. The exact physical location, connectivity, and configuration of these computing devices can be unknown and/or unimportant to client devices. Accordingly, the computing devices can be referred to as “cloud-based” devices that can be housed at various remote data center locations, such as a cloud-based server cluster. Desirably, the computing device is a cloud-based server cluster and inputting the concentration data to the decision tree ensemble is conducted via a web-based user interface where users can get access.

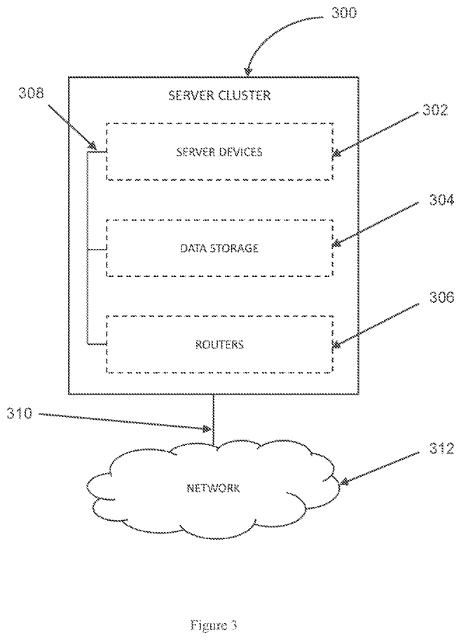

FIG. 3 depicts a schematic drawing of a cloud-based server cluster 300 in accordance with one example of the present invention. Desirably, operations of a computing device can be distributed between server devices 302, data storage 304, and routers 306, all of which can be connected by local cluster network 308. The amount of server devices 302, data storage 304, and routers 306 in the server cluster 300 can depend on the computing task(s) and/or applications assigned to the server cluster 300. For example, the server devices 302 can be configured to perform various computing tasks of the computing device. Thus, computing tasks can be distributed among one or more of the server devices 302. As an example, the data storage 304 can store any form of database, such as a structured query language (SQL) database or trained model checkpoints. Furthermore, any databases in the data storage 304 can be monolithic or distributed across multiple physical devices. The routers 306 can include networking equipment configured to provide internal and external communications for the server cluster 300. For example, the routers 306 can include one or more packet-switching and/or routing devices (including switches and/or gateways) configured to provide (i) network communications between the server devices 302 and the data storage 304 via the cluster network 308, and/or (ii) network communications between the server cluster 300 and other devices via the communication link 310 to the network 312. The server devices 302 can be configured to transmit data to and receive data from cluster data storage 304. Furthermore, server devices 302 can organize the received data into web page representations. Such a representation can take the form of a markup language, such as the hypertext markup language (HTML), the extensible markup language (XML), or some other standardized or proprietary format. Moreover, the server devices 302 can have the capability of executing various types of computerized scripting languages, such as Perl, Python, PHP Hypertext Preprocessor (PHP), Active Server Pages (ASP), or JavaScript. Computer program code written in these languages can facilitate the providing of web pages to client devices, as well as client device interaction with the web pages.

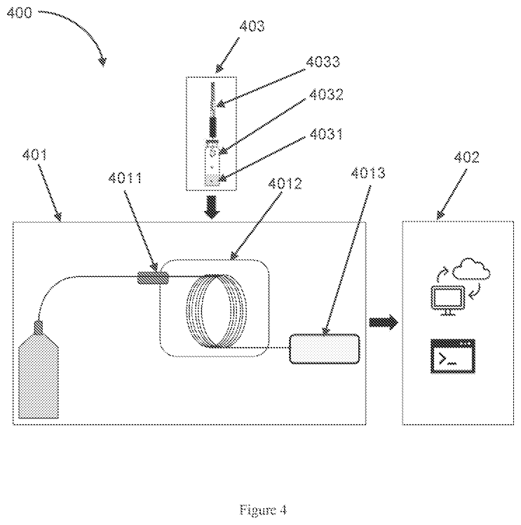

FIG. 4 illustrates a schematic block diagram showing an odor prediction system 400 in accordance with one example of the present invention. Desirably, the odor prediction system 400 comprises a GC-MS 401 to determine concentrations of volatile organic compounds (VOCs) in a sample, and a cloud-based computing platform 402 such as a cloud-based server cluster. Trained decision tree ensemble models are deployed in the cloud-based computing platform 402 to provide a web-based access for end users. The GC-MS 401 comprises a sample injector 4011, a GC column 4012, and an MSD 4013. A sample is first analytically characterized using the GC-MS 401 to generate VOC concentration data from the sample and the resulting concentration data is then inputted into the cloud-based computing platform 402. The odor prediction system 400 can further comprise a sampling unit 403 comprising a headspace vial 4032 and a SPME fiber 4033. Desirably, a sample 4031 (e.g., the aqueous polymer composition or coating) is added into the headspace vial 4032 and then the SPME fiber 4033 is inserted into the headspace vial 4032 and exposed to the headspace of the vial to extract the VOCs in the sample. The VOCs extracted from the sample 4031 are then injected into the sample injector 4011 and analyzed in the GC-MS 401 to generate VOC profiles. The obtained VOC profiles are used as the inputs and uploaded to the cloud-based computing platform 402 deployed with the models via a web-based user interface. Then the models predict the odor intensity of each sample based on the VOC profiles of such sample.

### Examples

Some embodiments of the invention will now be described in the following Examples. PRIMAL, FORMASHIELD, and RHOPLEX are all trademarks of The Dow Chemical Company.

Methyl methacrylate, butyl acetate, xylene (containing 3 isomers), butyl ether, styrene, butyl acrylate, propanoic acid, butyl ester, 3-methyl-4-heptone, and benzaldehyde are available from Sinopharm Chemical Reagent Co., Ltd.

Thirty-nine aqueous acrylic binders, commercially available from various sources (including PRIMAL™, FORMASHIELD™, and RHOPLEX™ emulsions available from The Dow Chemical Company, ACRONAL emulsions available from BASF, ARCHSOL™ emulsions available from Wanhua, and RS series emulsions available from BATF), are used as training and validation samples with 31 samples as training samples and 8 samples as validation samples, chosen in a random manner. These samples are randomly labeled with numbers “1” to “39” as sample codes in Tables 2-4.

Another six acrylic binders, commercially available from various sources as described above for the training samples and validation samples, are used as new samples to test models and randomly labeled with numbers “41” through “46” as sample codes in Table 5.

PRIMAL™ SF-180 emulsion containing a 100% acrylic polymer, PRIMAL™ DC-430V styrene acrylic copolymer emulsion, and PRIMAL™ SF-508M styrene acrylic emulsion are all commercially available from The Dow Chemical Company.

The following standard analytical equipment and methods are used in the Examples and in determining the properties and characteristics stated herein:

SPME Coupled with GC-MS Analysis

### 1) Preparation of External Standards

A standard mixture was prepared by mixing methyl methacrylate, butyl acetate, xylene (containing 3 isomers), butyl ether, styrene, butyl acrylate, propanoic acid, butyl ester, 3-methyl-4-heptone, and benzaldehyde with PRIMAL™ SF-180 emulsion, and each of these chemical compounds was present at a concentration of 10,000 ppm of the wet weight of PRIMAL™ SF-180 emulsion. The prepared standard mixture was further diluted to different concentrations for each chemical compound, such as 0.005 ppm, 0.01 ppm, 0.05 ppm, 0.1 ppm, 0.5 ppm, 1 ppm, 5 ppm, 10 ppm, 50 ppm, 100 ppm, and 500 ppm, based on the wet weight of PRIMAL™ SF-180 emulsion, for use as external standards for quantification of different VOCs.

### 2) Automated SPME GC-MS Test Parameters

Around 0.05 g of an aqueous binder sample was weighed into a 20 mL headspace vial.

A multipurpose sampler (MPS) (Gerstel) with a SPME unit is coupled to GC. The SPME fiber, available from Anple, is polydimethylsiloxane/divinylbenzene (PDMS/DVB, 65 millimeters (mm), Cat. 57345-U, Supelco Co., Ltd.). Parameters for SPME treatment are as follows: incubation temperature: 60° C., extraction time: 30 minutes, and desorption time: 90 seconds.

GC-MS analysis was conducted using an Agilent 6890 gas chromatograph coupled with a mass spectrometric detector (Agilent 5975C MSD) based on conditions listed in Table 1.

TABLE 1
GC-MS Conditions

| | |
|---|---|
| Injector Temperate | 250° C. |
| Oven Program-Initial Temperature | 50° C. |
| Initial Time | 4 minutes (min) |
| Temperature Ramp | 16° C./min to 250° C. for 2 min |
| Column Type | DB-5 ms 350° C.: 30 meters (m) × 250 mm × 0.25 mm |
| MS Detector | 29-400 Da, MS Source: 230° C.; MS Quad: 150° C. |
| Single Ion Monitoring (SIM) | Ions selected for each VOC for quantification: |
| Parameters | Acetone, 2-methy propanol: 58, 59 m/z (mass-to-charge ratio) |
| | Acetic acid: 60 m/z |
| | Ethyl acetate: 88 m/z |
| | 1-Butanol: 56 m/z |
| | Methyl isobutyrate: 87 m/z |
| | Ethyl acrylate: 99 m/z |
| | Methyl methacrylate and propanoic acid, ethyl ester: 100, 102 m/z |
| | Butanoic acid, ethyl ester: 100, 102 m/z |
| | Butyl acetate: 73 m/z |
| | 4-Heptanone: 114 m/z |
| | Ethylbenzene: 91 m/z |
| | Butyl ether, 2-heptanone, styrene, xylene, and butyl acrylate: 73, 87, 91, 104, 114 m/z |
| | Propanoic acid, butyl ester and phenol: 75, 94 m/z |
| | Anisole: 108 m/z |
| | Methyl ethyl benzene, 3-methyl-4-heptanone: 105, 128 m/z |
| | Propenyl benzene: 118 m/z |
| | Propyl benzene: 91 m/z |
| | Benzaldehyde: 105 m/z |
| | Acetophenone: 112, 120 m/z |
| | Butyl methacrylate, isobutyl vinylacetate: 86, 87 m/z |
| | Butanoic acid, butyl ester: 89 m/z |
| | 2-Butenoic acid, butyl ester: 69 m/z |
| | Diethyl benzene isomers1 and 2: 134 m/z |
| | Cyclohexyl methacrylate: 87 m/z |
| | 2-Ethylhexyl acrylate: 70 m/z |

### Odor Panel Testing

Testing was conducted according to the VDA 270 standard with experimental procedures and sample preparation described as follows:

In one sensory test, three test samples were prepared for evaluation by each panelist. Two benchmarking samples, PRIMAL™ DC-430V and PRIMAL™ SF-508M, were used in each sensory test for reference. The odor intensity values for PRIMAL™ SF-508M and PRIMAL™ DC-430V emulsions are rated as ‘1.5’ and ‘5.0’, respectively, according to the criteria described in the VDA 270 standard below.

An aliquot of 0.5 g of a binder sample was put into a 100 mL glass vial with an odorless cap. The vial with the sample was equilibrated at room temperature for 2 hours before evaluation. Each panelist independently received one set of three samples. A label, chosen at random, was assigned to each sample for blind sample identification. The order in which the samples were presented to panelists was also randomized.

For each test, eight to ten well-trained panelists (certified by SGS. Co., Ltd. on odor intensity training) were invited to make sensory evaluation of these samples. The variance analysis of sensory evaluation was calculated by CSAS (Conventional Sensory Analysis System) sensory software (ISENSO Co., Ltd., Shanghai, China) automatically.

The odor intensity is evaluated and rated based on the criteria described in the VDA 270 standard (the smallest unit is 0.5): 1. Not perceptible; 2. Perceptible, not disturbing; 3. Clearly perceptible, but not disturbing; 4. Disturbing; 5. Strongly disturbing; 6. Not acceptable.

The average value of the odor intensity rated by the panelists is reported for each binder sample, denoted as “actual odor intensity”.

### Models

The Random Forest (RF) model, Gradient Boosting (GB) model, Artificial Neural Network (ANN) model, Partial Least Square Regression (PLS) model and Ridge Regression (RR) model used in the following examples (IEs 1-3 and CEs 1-3) below were each independently constructed based on the Python Scikit-learn (Further details can be found in “Scikit-learn: Machine Learning in Python”, Pedregosa, F. et al., Journal of Machine Learning Research, 2011, 12, 2825-2830.) These models were each independently built with Scikit-learn 0.23.2 in a Python 3.8 environment.

The eXtreme Gradient Boosting (XGB) model was built with the XGBoost Package version 1.4.2 with Python 3.8 environment (Further details can be found in “XGBoost: A Scalable Tree Boosting System”, Chen, T., and Guestrin, C., Proceedings of the 22^{nd} ACM SIGKDD International Conference on Knowledge Discovery and Data Mining, 2016, 785-794, ACM, New York, USA).

Each model was trained with a total of 39 pairs of data from training samples, which was split into a training dataset with 31 data pairs (dataset type: “training”) and a validation dataset with 8 data pairs (dataset type: “validation”). The inputs to the model were concentrations of 27 selected VOCs as measured by GC-MS, and the output of the model was the odor intensity of the training samples rated by human panelists according to the odor panel testing described above. Results are given in Tables 2-4. To verify the effectiveness and accuracy of the method using each model, VOC concentrations for six new samples (i.e., test samples) were measured and inputted to each trained model. The predicted odor intensity values were obtained and are compared with manual evaluation results of odor intensity rated by human panelists in Table 5.

### IE-1

An automated odor intensity prediction tool was established by combining the SPME GC-MS, the odor panel testing, and a RF model. Hyperparameters of the RF model are shown below:

Number of estimators=100; Minimum number of samples per split=2; Minimum samples per leaf node=1; Minimum weight fraction of a leaf node=0; Maximum of features for split=\sqrt{n}; and Minimum of impurity decrease=0.

### IE-2

IE-2 was conducted according to the same procedure as IE-1 except the RF model was replaced with a GB model with the hyperparameters shown below:

Learning rate=0.1; Number of estimators=100; Fraction of samples to be used for individual base learners=1; Minimum number of samples per split=2; Minimum samples per leaf node=1; Minimum weight fraction of a leaf node=0; Maximum of depth of individual tree=3, and Minimum of impurity decrease=0.

### IE-3

IE-3 was conducted according to the same procedure as IE-1 except the RF model was replaced with an XGB model with the hyperparameters shown below:

Step size shrinkage=0.3; Minimum of impurity decrease=0; Maximum of depth of individual tree=6; Minimum sum of instance weight=1; Maximum delta step=0; Minimum samples per leaf node=1; Co-sample by tree, co-sample by leaf, co-sample by node=1; L2 regularization term on weight=1; and L1 regularization term on weight=0.

### CE-1

CE-1 was conducted according to the same procedure as IE-1 except the RF model was replaced with an ANN model with the hyperparameters shown below:

Hidden layer size=100, activation=ReLU (rectified linear unit), solver=Adam, alpha=0.0001, batch size=number of samples, learning rate=0.001, maximum number of iterations=200, shuffle=True, early stopping=False, beta1=0.9, beta2=0.999, epsilon=10⁻⁸.

### CE-2

CE-2 was conducted according to the same procedure as IE-1 except the RF model was replaced with a PLS model with the hyperparameters shown below: Number of components=2, scale=true, maximum number of iterations=500, tolerance=10⁻⁶, copy=true.

### CE-3

CE-3 was conducted according to the same procedure as IE-1 except the RF model was replaced with an RR model with the hyperparameters shown below:

Alpha=0.2, fit intercept=true, normalize=false, copy X=true, maximum number of iterations=15000, tolerance=10⁻³, solver=auto, positive=false.

The raw data (including VOC concentrations and actual odor intensity) and model predictions for training samples and validation samples according to the methods described in the above examples (IE-1 to IE-3 and CE-1 to CE-3) are presented in Tables 2-4. Table 5 gives the odor evaluation results for test samples. In these tables, the actual odor intensity refers to the odor intensity rated by panelists according to the odor panel testing described above, and “\” means the concentration is below the detection limit.

TABLE 2
Odor evaluation for training and validation samples

| Sample Code | | | | | | | | | | | | |
|---|---|---|---|---|---|---|---|---|---|---|---|---|
| Sam- | Sam- | Sam- | Sam- | Sam- | Sam- | Sam- | Sam- | Sam- | Sam- | Sam- | Sam- | Sam- |
| ple 1 | ple 30 | ple 29 | ple 23 | ple 31 | ple 22 | ple 21 | ple 20 | ple 24 | ple 39 | ple 17 | ple 18 | ple 19 |

VOC concentration (ppm)

| | | | | | | | | | | | | | |
|---|---|---|---|---|---|---|---|---|---|---|---|---|---|
| Acetone | 0.2 | \ | \ | 9.5 | \ | 10.1 | 8.8 | 12.4 | 6.4 | 0.2 | 1.8 | 2.1 | 2.1 |
| 2-Methyl propanol | 0.7 | 0.4 | 0.3 | 6.4 | 0.5 | 6.3 | 5.8 | 7.8 | 5.9 | 0.9 | 7.6 | 9.0 | 9.2 |
| 1-Butanol | 4.1 | 1.5 | 2.5 | 3.4 | 2.0 | 3.3 | 3.5 | 3.9 | 9.1 | 1.5 | 6.0 | 6.7 | 6.2 |
| Methyl methacrylate | 1.6 | \ | \ | 0.2 | \ | \ | \ | \ | \ | \ | 0.3 | \ | 0.3 |
| Butyl acetate | 11.5 | 0.9 | 1.8 | 9.8 | 1.1 | 6.2 | 5.6 | 2.3 | 34.0 | 11.6 | 16.0 | 18.3 | 17.7 |
| 2-Heptanone | \ | \ | \ | \ | \ | \ | \ | \ | 0.3 | 0.2 | \ | \ | \ |
| Butyl ether | 4.1 | 0.4 | 0.9 | 1.3 | 1.1 | 1.1 | 0.8 | 0.4 | 5.9 | 24.1 | 1.6 | 2.1 | 2.2 |
| Styrene | \ | \ | 0.2 | 1.1 | 0.3 | 1.1 | 0.9 | 1.1 | 1.1 | 0.6 | 0.8 | 0.5 | 0.8 |
| Butyl acrylate | 0.9 | 1.0 | 2.3 | 5.8 | 6.6 | 7.9 | 8.6 | 12.0 | 15.9 | 20.8 | 33.4 | 42.8 | 65.9 |
| Propanoic acid, butyl | 65.9 | 3.9 | 2.4 | 5.1 | 5.9 | 6.0 | 4.6 | 4.8 | 15.9 | 10.4 | 6.4 | 7.8 | 7.9 |
| ester | | | | | | | | | | | | | |
| Anisole | \ | \ | \ | \ | \ | 0.2 | \ | 0.2 | \ | \ | \ | \ | \ |
| Methyl ethyl benzene | 14.8 | 2.0 | 2.8 | 3.4 | 3.7 | 5.2 | 4.1 | 3.4 | 5.2 | 7.8 | 2.2 | 4.6 | 2.4 |
| 3-Methyl-4-heptanone | \ | \ | 0.3 | 0.3 | \ | 0.2 | 0.2 | \ | 2.0 | 1.2 | 0.2 | 0.3 | 0.3 |
| Propenyl benzene | 1.2 | 0.2 | 0.3 | 0.4 | 0.5 | 0.6 | 0.5 | 0.3 | 0.4 | 0.8 | 0.2 | 0.5 | 0.2 |
| Propyl benzene | 19.0 | 1.6 | 3.1 | 3.2 | 4.8 | 5.9 | 4.1 | 2.8 | 6.6 | 7.3 | 2.3 | 3.1 | 2.1 |
| Benzaldehyde | 38.1 | 27.8 | 22.9 | 22.8 | 31.3 | 23.2 | 19.7 | 17.9 | 25.2 | 25.9 | 11.6 | 15.7 | 15.6 |
| Butyl methacrylate | 0.4 | \ | \ | \ | \ | \ | \ | \ | 0.2 | \ | \ | \ | \ |
| Isobutyl vinylacetate | 0.3 | \ | \ | \ | \ | \ | \ | \ | 0.2 | \ | \ | \ | \ |
| Butanoic acid, butyl ester | 56.5 | 14.2 | 57.4 | 40.8 | 22.6 | 33.7 | 30.8 | 6.0 | 224.8 | 191.2 | 28.7 | 43.2 | 34.6 |
| 2-Butenoic acid, butyl | 1.2 | 1.5 | 3.9 | 2.1 | 2.2 | 1.4 | 1.3 | \ | 8.2 | 8.7 | 2.2 | 4.6 | 6.2 |
| ester | | | | | | | | | | | | | |
| Cyclohexyl methacrylate | 0.6 | \ | \ | 0.1 | \ | \ | \ | \ | \ | \ | \ | \ | \ |
| 2-Ethylhexyl acrylate | 1.8 | 82.7 | 367.1 | 17.4 | 890.0 | 12.0 | 8.2 | 15.7 | 15.4 | 1023.1 | 8.2 | 8.5 | 6.1 |
| Xylene or ethyl benzene | 21.5 | 13.8 | 11.0 | 12.2 | 9.8 | 5.8 | 4.6 | 10.2 | 5.8 | 13.7 | 6.3 | 9.9 | 9.2 |
| Total VOC concentration | 244.4 | 151.8 | 479.1 | 145.2 | 982.3 | 130.0 | 112.2 | 101.2 | 388.5 | 1349.9 | 135.8 | 179.5 | 189.0 |

Odor Intensity Score and Model Predictions

| Actual Odor Intensity | | | 2.8 | 2.3 | 3.2 | 4.7 | 3.7 | 4.0 | 4.8 | 5.0 | 4.6 | 4.7 | 5.6 | 4.9 | 5.6 |
|---|---|---|---|---|---|---|---|---|---|---|---|---|---|---|---|
| Dataset Type | | | Train- | Valida- | Train- | Train- | Valida- | Train- | Train- | Train- | Train- | Train- | Train- | Train- | Valida- |
| | | | ing | tion | ing | ing | tion | ing | ing | ing | ing | ing | ing | ing | tion |
| | Exam- | Model | | | | | | | | | | | | | |
| | ple | Type | | | | | | | | | | | | | |
| Pre- | CE-1 | ANN | 3.3 | 1.7 | 2.4 | 4.6 | 3.8 | 4.8 | 4.4 | 5.1 | 5.0 | 5.3 | 4.4 | 4.9 | 5.7 |
| dict- | CE-2 | PLS | 2.7 | 2.5 | 2.9 | 4.6 | 3.8 | 5.2 | 4.6 | 5.4 | 5.1 | 4.7 | 4.8 | 5.3 | 6.2 |
| ed | IE-1 | RF | 3.0 | 2.3 | 2.8 | 4.6 | 2.7 | 4.3 | 4.7 | 4.9 | 4.5 | 4.2 | 5.2 | 4.9 | 5.1 |
| Odor | IE-2 | GB | 2.8 | 3.0 | 3.2 | 4.7 | 3.1 | 4.0 | 4.8 | 5.0 | 4.6 | 4.7 | 5.6 | 4.9 | 5.4 |
| Inten- | IE-3 | XGB | 2.8 | 2.9 | 3.2 | 4.7 | 3.2 | 4.0 | 4.8 | 5.0 | 4.6 | 4.7 | 5.6 | 4.9 | 5.3 |
| sity | CE-3 | RR | 2.8 | 2.6 | 2.9 | 4.5 | 3.5 | 4.5 | 4.3 | 4.6 | 4.9 | 4.2 | 4.3 | 4.5 | 5.0 |

TABLE 3
Odor evaluation for training and validation samples

| Sample Code | | | | | | | | | | | | |
|---|---|---|---|---|---|---|---|---|---|---|---|---|
| Sam- | Sam- | Sam- | Sam- | Sam- | Sam- | Sam- | Sam- | Sam- | Sam- | Sam- | Sam- | Sam- |
| ple 25 | ple 28 | ple 27 | ple 26 | ple 3 | ple 2 | ple 32 | ple 9 | ple 10 | ple 33 | ple 8 | ple 35 | ple 36 |

VOC Concentration (ppm)

| | | | | | | | | | | | | | |
|---|---|---|---|---|---|---|---|---|---|---|---|---|---|
| Acetone | \ | \ | \ | \ | \ | \ | 0.5 | 1.8 | 1.9 | 0.3 | 3.0 | 2.8 | 3.2 |
| 2-Methyl propanol | 0.2 | \ | \ | \ | 0.3 | 0.3 | 4.1 | 0.4 | 0.3 | 1.9 | \ | 2.5 | 18.9 |
| 1-Butanol | 2.6 | 1.4 | 1.6 | 2.3 | 1.8 | 2.4 | 6.7 | 4.3 | 5.0 | 5.2 | 7.7 | 7.7 | 25.6 |
| Methyl methacrylate | \ | \ | \ | \ | 1.6 | 1.1 | \ | \ | \ | \ | \ | \ | \ |
| Butyl acetate | 0.4 | 1.1 | 0.4 | 0.5 | 7.7 | 7.3 | 4.7 | 1.8 | 4.4 | 14.3 | 1.5 | 5.8 | 41.0 |
| 4-Heptanone | \ | \ | \ | \ | \ | \ | \ | \ | \ | \ | \ | \ | 0.5 |
| 2-Heptanone | \ | \ | \ | \ | \ | \ | \ | \ | \ | \ | \ | 0.6 | 0.4 |
| Butyl ether | 0.3 | 0.4 | 0.2 | 0.4 | 3.8 | 3.3 | 4.4 | 6.7 | 3.8 | 3.0 | 5.6 | 132.4 | 16.8 |
| Styrene | \ | 0.2 | \ | 0.5 | \ | 0.2 | \ | \ | \ | \ | \ | \ | 0.6 |
| Butyl acrylate | 0.2 | 0.3 | 0.2 | 0.8 | 0.4 | 0.5 | \ | \ | \ | \ | \ | \ | 1.4 |
| Propanoic acid, butyl | 1.7 | 1.8 | 0.7 | 1.9 | 48.5 | 45.4 | 15.7 | 14.9 | 16.9 | 17.8 | 19.8 | 6.7 | 29.5 |
| ester | | | | | | | | | | | | | |
| Methyl ethyl benzene | 2.6 | 4.3 | 1.6 | 3.2 | 5.0 | 4.3 | 5.3 | 3.0 | 10.4 | 6.3 | 0.9 | 7.8 | 7.2 |
| 3-Methyl-4-heptanone | \ | \ | \ | \ | \ | \ | 0.3 | 0.5 | 0.9 | 0.5 | 0.4 | 1.9 | 0.8 |
| Propenyl benzene | 0.3 | 0.5 | 0.2 | 0.4 | 0.4 | 0.3 | 0.4 | \ | 0.3 | 0.6 | \ | 0.7 | 0.6 |
| Propyl benzene | 3.3 | 5.7 | 2.2 | 4.1 | 3.7 | 3.1 | 7.4 | 1.6 | 8.2 | 9.5 | 0.6 | 7.0 | 7.8 |
| Benzaldehyde | 13.4 | 19.3 | 14.0 | 18.1 | 29.4 | 23.8 | 34.4 | 0.5 | 1.5 | 33.7 | 0.3 | 2.6 | 51.0 |
| Acetophenone | \ | \ | \ | \ | \ | \ | \ | \ | \ | \ | \ | 0.3 | 0.5 |
| Butyl methacrylate | \ | \ | \ | \ | 0.3 | 0.2 | 0.5 | \ | \ | 0.8 | \ | 0.3 | 0.4 |
| Isobutyl vinylacetate | \ | \ | \ | \ | \ | \ | 0.2 | 0.2 | 0.3 | 0.3 | 0.3 | \ | \ |
| Butanoic acid, butyl ester | 5.5 | 7.7 | 20.0 | 15.4 | 40.0 | 38.1 | 85.6 | 162.7 | 163.3 | 98.5 | 181.5 | 85.1 | 359.7 |
| 2-Butenoic acid, butyl | 0.2 | 0.5 | 1.4 | 0.7 | 1.2 | 0.9 | 16.0 | 13.3 | 6.9 | 27.1 | 23.0 | 11.2 | 2.5 |
| ester | | | | | | | | | | | | | |
| Diethyl benzene isomer 1 | \ | \ | \ | \ | \ | \ | \ | \ | \ | \ | \ | 1.7 | \ |
| Diethyl benzene isomer 2 | \ | \ | \ | \ | \ | \ | \ | \ | \ | \ | \ | 0.7 | \ |
| Cyclohexyl methacrylate | \ | \ | \ | \ | 0.4 | 0.4 | \ | \ | \ | \ | \ | \ | \ |
| 2-Ethylhexyl acrylate | 8.7 | 14.9 | 31.0 | 59.7 | 1.2 | 2.4 | 4.8 | \ | \ | 7.4 | \ | 0.2 | \ |
| Xylene or ethyl benzene | 3.3 | 4.6 | 1.4 | 5.4 | 17.1 | 37.5 | 4.4 | 6.5 | 7.7 | 6.0 | 5.7 | 20.9 | 73.1 |
| Total VOC concentration | 39.5 | 60.4 | 72.8 | 110.7 | 151.4 | 160.3 | 179.3 | 209.8 | 220.3 | 211.4 | 237.9 | 280.0 | 552.7 |

Odor Intensity Score and Model Predictions

| Actual Odor Intensity | | | 2.8 | 2.8 | 1.9 | 1.9 | 2.1 | 1.9 | 3.2 | 1.4 | 2.1 | 4.5 | 1.4 | 2.8 | 5.0 |
|---|---|---|---|---|---|---|---|---|---|---|---|---|---|---|---|
| Dataset Type | | | Valida- | Valida- | Train- | Train- | Train- | Train- | Train- | Train- | Train- | Train- | Train- | Valida- | Train- |
| | | | tion | tion | ing | ing | ing | ing | ing | ing | ing | ing | ing | tion | ing |
| | Exam- | Model | | | | | | | | | | | | | |
| | ple | Type | | | | | | | | | | | | | |
| Pre- | CE-1 | ANN | 1.4 | 1.8 | 1.3 | 2.2 | 1.9 | 2.1 | 2.9 | 1.9 | 2.3 | 3.3 | 2.2 | 3.5 | 5.0 |
| dict- | CE-2 | PLS | 2.4 | 2.7 | 2.3 | 2.9 | 2.1 | 2.1 | 3.3 | 2.2 | 2.4 | 3.4 | 2.3 | 2.8 | 4.9 |
| ed | IE-1 | RF | 2.0 | 2.0 | 2.0 | 2.2 | 2.1 | 2.1 | 3.6 | 1.6 | 2.0 | 4.2 | 1.6 | 3.7 | 4.7 |
| Odor | IE-2 | GB | 1.9 | 1.9 | 1.9 | 1.9 | 2.1 | 1.9 | 3.2 | 1.4 | 2.1 | 4.5 | 1.4 | 3.7 | 5.0 |
| Inten- | IE-3 | XGB | 1.9 | 1.9 | 1.9 | 1.9 | 2.1 | 1.9 | 3.2 | 1.4 | 2.1 | 4.5 | 1.4 | 4.0 | 5.0 |
| sity | CE-3 | RR | 2.4 | 2.7 | 2.4 | 3.0 | 2.3 | 2.4 | 3.3 | 2.4 | 2.6 | 3.5 | 2.4 | 3.2 | 5.0 |

TABLE 4
Odor evaluation for training and validation samples

| Sample Code | | | | | | | | | | | | |
|---|---|---|---|---|---|---|---|---|---|---|---|---|
| 15 | 16 | 38 | 34 | 14 | 37 | 6 | 4 | 7 | 13 | 11 | 5 | 12 |

VOC Concentration (ppm)

| | | | | | | | | | | | | | |
|---|---|---|---|---|---|---|---|---|---|---|---|---|---|
| Acetone | 2.3 | 2.8 | \ | 0.4 | 2.4 | 0.2 | 0.4 | 0.4 | 2.9 | 1.2 | 2.4 | 0.3 | 1.9 |
| 2-Methyl propanol | 1.4 | 2.2 | 0.2 | 4.0 | 2.7 | 2.0 | 3.7 | 4.3 | 16.7 | 2.1 | 3.3 | 3.3 | 2.8 |
| 1-Butanol | 5.0 | 5.7 | 7.9 | 7.7 | 7.2 | 9.2 | 6.7 | 6.9 | 11.0 | 14.6 | 25.3 | 6.0 | 19.0 |
| Butyl acetate | 32.6 | 34.8 | 5.9 | 25.0 | 37.5 | 32.8 | 53.0 | 50.7 | 82.6 | 53.3 | 67.9 | 53.4 | 77.5 |
| 2-Heptanone | 0.2 | 0.2 | \ | \ | 0.3 | 0.3 | 0.5 | 0.5 | 0.3 | 0.4 | 0.7 | 0.7 | 0.6 |
| Butyl ether | 15.5 | 14.7 | 34.2 | 7.0 | 26.1 | 39.0 | 13.2 | 11.2 | 21.3 | 19.5 | 13.6 | 14.3 | 13.9 |
| Propanoic acid, butyl | 38.8 | 43.5 | 9.1 | 36.6 | 76.4 | 23.2 | 43.6 | 55.6 | 110.5 | 73.8 | 82.7 | 47.4 | 70.2 |
| ester | | | | | | | | | | | | | |
| Methyl ethyl benzene | 4.1 | 5.2 | 9.7 | 10.2 | 6.2 | 15.8 | 10.1 | 15.7 | 8.6 | 6.2 | 10.3 | 13.0 | 12.5 |
| 3-Methyl-4-heptanone | 1.2 | 1.7 | 1.4 | 1.0 | 1.1 | 2.4 | 2.8 | 2.9 | 2.6 | 2.8 | 2.7 | 3.4 | 3.5 |
| Propenyl benzene | 0.2 | 0.2 | 0.7 | 0.9 | 0.5 | 0.7 | 0.6 | 0.9 | 0.4 | 0.4 | 0.9 | 0.7 | 0.9 |
| Propyl benzene | 2.8 | 2.1 | 9.3 | 12.7 | 5.8 | 15.9 | 10.0 | 15.7 | 5.6 | 6.2 | 12.9 | 13.3 | 10.4 |
| Benzaldehyde | 1.0 | 0.3 | 18.7 | 46.2 | 1.8 | 29.3 | 48.3 | 56.8 | 24.5 | 1.7 | 2.6 | 52.8 | 2.3 |
| Acetophenone | \ | \ | \ | \ | 0.2 | \ | \ | \ | \ | 0.2 | 0.2 | \ | \ |
| Butyl methacrylate | \ | \ | 0.2 | 1.0 | 0.2 | 0.2 | 0.2 | \ | \ | 0.2 | 0.3 | \ | 0.3 |
| Isobutyl vinylacetate | 0.5 | 0.3 | \ | 0.5 | 0.3 | 0.2 | 0.2 | 0.2 | 0.6 | 0.3 | \ | 0.2 | 0.3 |
| Butanoic acid, butyl ester | 213.5 | 226.2 | 284.4 | 190.0 | 268.2 | 367.1 | 530.6 | 514.0 | 459.3 | 585.6 | 582.9 | 660.3 | 692.3 |
| 2-Butenoic acid, butyl | 29.5 | 15.7 | 7.1 | 61.8 | 12.0 | 12.0 | 16.6 | 17.8 | 27.0 | 53.6 | 32.7 | 16.4 | 31.9 |
| ester | | | | | | | | | | | | | |
| Diethyl benzene isomer 1 | \ | \ | 0.9 | \ | 1.2 | \ | \ | \ | 0.5 | \ | \ | \ | \ |
| Diethyl benzene isomer 2 | \ | \ | 0.3 | \ | 0.5 | \ | \ | \ | 0.2 | \ | \ | \ | \ |
| 2-Ethylhexyl acrylate | \ | \ | 0.2 | 2.8 | \ | 0.2 | \ | \ | \ | \ | \ | \ | \ |
| Xylene or ethyl benzene | 17.3 | 18.9 | 20.1 | 12.7 | 17.1 | 34.3 | 33.2 | 32.1 | 26.8 | 18.1 | 15.1 | 29.3 | 27.6 |
| Total VOC | 365.9 | 374.6 | 410.4 | 420.6 | 467.8 | 584.9 | 773.5 | 785.7 | 801.6 | 840.3 | 856.3 | 915.0 | 967.7 |
| concentrations | | | | | | | | | | | | | |

Odor Intensity Score and Model Predictions

| Actual Odor Intensity | | | 3.5 | 4.0 | 1.8 | 4.3 | 3.8 | 3.6 | 3.4 | 3.0 | 5.3 | 3.1 | 3.6 | 3.4 | 2.4 |
|---|---|---|---|---|---|---|---|---|---|---|---|---|---|---|---|
| Dataset Type | | | Train- | Train- | Train- | Train- | Train- | Train- | Train- | Valida- | Train- | Train- | Valida- | Train- | Train- |
| | | | ing | ing | ing | ing | ing | ing | ing | tion | ing | ing | tion | ing | ing |
| | Exam- | Model | | | | | | | | | | | | | |
| | ple | Type | | | | | | | | | | | | | |
| Pre- | CE-1 | ANN | 3.3 | 2.8 | 2.1 | 4.7 | 3.4 | 3.4 | 3.2 | 3.8 | 5.5 | 3.3 | 2.7 | 3.0 | 3.2 |
| dict- | CE-2 | PLS | 2.8 | 2.7 | 2.5 | 4.1 | 3.0 | 2.9 | 3.3 | 3.5 | 4.6 | 2.8 | 3.0 | 3.2 | 3.1 |
| ed | IE-1 | RF | 3.7 | 3.9 | 1.9 | 4.0 | 3.7 | 3.6 | 3.4 | 3.4 | 4.6 | 3.2 | 3.3 | 3.4 | 2.8 |
| Odor | IE-2 | GB | 3.5 | 4.0 | 1.8 | 4.2 | 3.8 | 3.6 | 3.4 | 3.5 | 5.2 | 3.1 | 3.2 | 3.4 | 2.4 |
| Inten- | IE-3 | XGB | 3.5 | 4.0 | 1.8 | 4.2 | 3.8 | 3.6 | 3.4 | 3.3 | 5.2 | 3.1 | 2.6 | 3.4 | 2.4 |
| sity | CE-3 | RR | 3.1 | 3.0 | 2.5 | 4.2 | 3.4 | 3.1 | 3.5 | 3.7 | 4.9 | 3.1 | 3.1 | 3.4 | 3.3 |

TABLE 5
Odor Evaluation for Test Samples

| Sample Code | | | | | |
|---|---|---|---|---|---|
| Sam- | Sam- | Sam- | Sam- | Sam- | Sam- |
| ple 41 | ple 42 | ple 43 | ple 44 | ple 45 | ple 46 |

VOC Concentration (ppm)

| | | | | | | |
|---|---|---|---|---|---|---|
| Acetone | \ | 0.9 | 0.6 | 0.6 | 0.5 | 0.6 |
| 2-Methyl propanol | 0.7 | 6.7 | \ | \ | \ | \ |
| 1-Butanol | 3.2 | 7.8 | 1.7 | 1.8 | 2.2 | 2.3 |
| Butyl acetate | 43.0 | 30.8 | 10.4 | 5.1 | 18.0 | 10.2 |
| 2-Heptanone | \ | 0.4 | \ | \ | 0.2 | \ |
| Butyl ether | 103.1 | 110.1 | 47.2 | 42.4 | 92.1 | 82.5 |
| Styrene | 1.2 | 0.9 | \ | \ | \ | \ |
| Butyl acrylate | \ | 0.8 | 2.7 | 1.9 | 3.0 | 2.1 |
| Propanoic acid, butyl ester | 128.9 | 70.6 | 43.2 | 31.2 | 76.9 | 59.3 |
| Methyl ethyl benzene | 8.5 | 7.0 | 6.4 | 6.3 | 7.0 | 6.8 |
| 3-Methyl-4-heptanone | 0.9 | 0.7 | 1.0 | 0.8 | 2.0 | 1.6 |
| Propenyl benzene | 1.1 | 0.7 | 0.6 | 0.6 | 0.6 | 0.5 |
| Benzaldehyde | 23.3 | 26.3 | \ | \ | \ | \ |
| Butyl methacrylate | 0.4 | 0.3 | \ | \ | \ | \ |
| Isobutyl vinylacetate | 4.2 | \ | \ | \ | 0.2 | 0.2 |
| Butanoic acid, butyl ester | 120.0 | 67.9 | 89.8 | 78.0 | 157.6 | 138.3 |
| 2-Butenoic acid, butyl ester | 296.7 | 3.0 | 17.1 | 14.4 | 27.8 | 24.1 |
| 2-Ethylhexyl acrylate | 0.2 | \ | 176.1 | 174.6 | 0.2 | 0.2 |
| Xylene or ethyl benzene | 25.9 | 48.2 | 16.4 | 14.1 | 16.5 | 14.4 |
| Total VOC concentration | 761.2 | 383.1 | 413.2 | 371.8 | 404.8 | 343.2 |

Odor Intensity Score and Model Predictions

| Actual Odor Intensity | | | 3.3 | 4.2 | 3.4 | 3.0 | 3.2 | 2.6 |
|---|---|---|---|---|---|---|---|---|
| | Examples | Model Type | | | | | | |
| Predicted | CE-1 | ANN | 2.4 | 0.8 | 0.5 | 0.4 | 0.6 | 0.5 |
| Odor | CE-2 | PLS | 1.1 | 0.7 | 0.4 | 0.4 | 0.4 | 0.4 |
| Intensity | IE-1 | RF | 2.7 | 4.5 | 2.5 | 2.4 | 2.5 | 2.5 |
| | IE-2 | GB | 1.8 | 4.8 | 3.0 | 2.9 | 3.1 | 3.0 |
| | IE-3 | XGB | 1.8 | 4.3 | 2.8 | 2.8 | 2.8 | 2.8 |
| | CE-3 | RR | 1.4 | 0.8 | 0.4 | 0.4 | 0.5 | 0.5 |

Based on the data given in Tables 2-5, training R², validation percentage RMSE and test percentage RMSE were calculated based on the equations (II) and (III) above, and results are given in Table 6. As shown in Table 6, the IE-1 (RF model), IE-2 (GB model), and IE-3 (XGB model) methods showed higher training R² than CE-1 (ANN model), CE-2 (PLS model), and CE-3 (RR model). The validation and testing results for IE-1 to IE-3 methods all met the requirement of percentage RMSE<30%, while all CE-1 to CE-3 methods provided test percentage RMSE higher than 30%. It indicates that the IE-1 to IE-3 methods, which are all based on decision tree ensembles, showed higher prediction accuracy than CE-1 to CE-3 based on the ANN, PLS, and RR models, respectively. Particularly, IE-1 with the RF model showed even higher prediction accuracy (test percentage RMSE=18.7%) than IE-2 (test percentage RMSE=21.3%) and IE-3 (test percentage RMSE=20.6%).

TABLE 6
Results for training R², validation percentage RMSE, and test percentage RMSE

| | | Training Samples and Validation Samples | | Test Samples |
|---|---|---|---|---|
| Examples | Model type | Training R² | Validation percentage RMSE | Test percentage RMSE |
| CE-1 | ANN | 0.730 | 28.4% | 135.0% |
| CE-2 | PLS | 0.770 | 12.3% | 45.8% |
| IE-1 | RF | 0.903 | 21.0% | 18.7% |
| IE-2 | GB | 0.931 | 25.0% | 21.3% |
| IE-3 | XGB | 0.906 | 27.9% | 20.6% |
| CE-3 | RR | 0.771 | 13.7% | 66.3% |

1. A method of predicting odor of an aqueous polymer composition, comprising:
analytically characterizing the aqueous polymer composition with a detector, thereby generating concentration data for volatile organic compounds in the aqueous polymer composition from the analytical characterization;
inputting the concentration data to a decision tree ensemble configured to predict an odor intensity of the aqueous polymer composition based on the concentration data; and
outputting a predicted odor intensity of the aqueous polymer composition from the decision tree ensemble;
wherein the decision tree ensemble is trained to predict the odor intensity of the aqueous polymer composition using a training dataset using a plurality of training samples, wherein the training dataset comprises the concentration data for volatile organic compounds in each training sample paired with the actual odor intensity data rated by human panelists for such training sample; thereby giving a trained decision tree ensemble;
wherein the decision tree ensemble exhibits a prediction accuracy indicated by test percentage root mean square error <30% and training coefficient of discrimination >0.85.

2. (canceled)

3. The method of claim 1, wherein the trained decision tree ensemble is validated with a validation dataset using a plurality of validation samples, wherein the validation dataset comprises the concentration data for volatile organic compounds in each validation sample paired with the actual odor intensity data rated by human panelists for such validation sample.

4. The method of claim 1, wherein the decision tree ensemble is selected from a Random Forest model, a Gradient Boosting model, or an extreme Gradient Boosting model.

5. The method of claim 21, wherein the training dataset comprises the concentration data of volatile organic compounds that each has a concentration ≥0.1 part per million, by weight based on the weight of the aqueous polymer composition.

6. The method of claim 1, wherein the concentration data input to the decision tree ensemble is conducted via a web-based user interface.

7. The method of claim 1, wherein the concentration data input to the decision tree ensemble is the concentrations of volatile organic compounds comprising acetone, 2-methyl propanol, 1-butanol, methyl methacrylate, butyl acetate, 4-heptanone, 2-heptanone, butyl ether, styrene, butyl acrylate, anisole, propanoic acid, butyl ester, methyl ethyl benzene, 3-methyl-4-heptanone, propenyl benzene, propyl benzene, benzaldehyde, acetophenone, butyl methacrylate, isobutyl vinylacetate, butanoic acid, butyl ester, 2-butenoic acid, butyl ester, diethyl benzene or isomers, cyclohexyl methacrylate, 2-ethylhexyl acrylate, xylene, ethyl benzene, or mixtures thereof.

8. The method of claim 1, wherein analytically characterizing the aqueous polymer composition comprises an analytical characterization selected from solid phase micro-extraction coupled with gas chromatography-mass spectroscopy, needle trap microextraction coupled with gas chromatography-mass spectroscopy, or Tenax absorbent cartridge coupled with gas chromatography-mass spectroscopy.

9. (canceled)

10. The method of claim 1, further comprising adjusting polymerization process for preparing the aqueous polymer composition based on the predicted odor intensity.

11. The method of claim 1, wherein the aqueous polymer composition comprises an acrylic (co)polymer.

12. A method of predicting odor of a coating, comprising:
analytically characterizing the coating with a detector, thereby generating concentration data for volatile organic compounds in the coating from the analytical characterization; wherein the coating is obtained by drying an aqueous polymer composition;
inputting the concentration data to a decision tree ensemble configured to predict an odor intensity of the coating based on the concentration data; and
outputting a predicted odor intensity of the coating from the decision tree ensemble;
wherein the decision tree ensemble is trained to predict the odor intensity of the coating using a training dataset using a plurality of training samples, wherein the training dataset comprises the concentration data for volatile organic compounds in each training sample paired with the actual odor intensity data rated by human panelists for such training sample; thereby giving a trained decision tree ensemble;
wherein the decision tree ensemble exhibits a prediction accuracy indicated by test percentage root mean square error <30% and training coefficient of discrimination >0.85.

13. A system for predicting odor of an aqueous polymer composition or a coating made therefrom, comprising:
a detector, configured to analytically characterize the aqueous polymer composition or the coating, thereby generating concentration data for volatile organic compounds from the analytical characterization; and
a computing device with a decision tree ensemble deployed thereon, configured to input the concentration data and output a predicted odor intensity of the aqueous polymer composition or the coating;
wherein the decision tree ensemble is trained to predict the odor intensity of the aqueous polymer composition or the coating using a training dataset using a plurality of training samples, wherein the training dataset comprises the concentration data for volatile organic compounds in each training sample paired with the actual odor intensity data rated by human panelists for such training sample; thereby giving a trained decision tree ensemble;
wherein the decision tree ensemble exhibits a prediction accuracy indicated by test percentage root mean square error <30% and training coefficient of discrimination >0.85.

14. (canceled)

15. The system of claim 13, wherein the computing device is a cloud-based server cluster and inputting the concentration data to the decision tree ensemble is conducted via a web-based user interface.
